(19) United States
(12) Patent Application Publication
(10) Pub. No.: US 2017/0172767 A1
(43) Pub. Date: Jun. 22, 2017

(54) STENTS FOR PLACEMENT IN AN ANATOMICAL PASSAGEWAY AND METHODS

(71) Applicant: Cook Medical Technologies LLC, Bloomington, IN (US)

(72) Inventors: Jacob Buenger, Deshler, OH (US); Rachel Martini, Cincinnati, OH (US); Wen Hong Neoh, Bloomington, IN (US); Andreas Shick, Grand Blanc, MI (US); Lynesha Sweeney, Bloomington, IN (US); Justin West, Tiffin, OH (US)

(21) Appl. No.: 14/972,875

(22) Filed: Dec. 17, 2015

(51) Int. Cl. A61F 2/88; A61F 2/844; A61F 2/962

(52) U.S. Cl. CPC A61F 2/885; A61F 2/962; A61F 2/844; A61F 2220/0016

(57) ABSTRACT

Stents for placement in an anatomical passageway are described. Methods of stenting in an anatomical passageway are also described, for example in a liver between a portal vein and a hepatic vein as in a transjugular intrahepatic portosystemic shunt procedure. These are example embodiments and are not intended to be limiting.

### CROSS REFERENCE TO RELATED APPLICATIONS

This application claims the benefit of U.S. Provisional Application Ser. No. 62/100,264, filed Jan. 6, 2015 entitled Stents for Placement in an Anatomical Passageway and Methods, which is hereby incorporated by reference in its entirety.

### FIELD

The disclosure relates to the field of stents and methods for use in anatomical passageways.

BACKGROUND

Normally blood flows from the esophagus, stomach, and intestines through the portal vein into the liver. A healthy liver processes the blood and absorbs nutrients from the gut. The blood then filters through the liver tissue and drains into the hepatic veins and then into the heart. When damage to the liver occurs there is an increased pressure in the network of veins that drain the stomach, esophagus, and intestines known as portal hypertension. The most common reason for this problem is cirrhosis of the liver. The current use of a covered, non-thrombogenic shunt creates a new path between the hepatic and portal veins to keep the blood from building up in the liver and alleviate pressure buildup. This procedure is known as Transjugular Intrahepatic Portosystemic Shunt (TIPS).

### SUMMARY

Stents for placement in an anatomical passageway are described. Methods of stenting in an anatomical passageway are also described, for example in a liver between a portal vein and a hepatic vein as in a transjugular intrahepatic portosystemic shunt procedure. These are examples and are not intended to be limiting.

### DESCRIPTION OF THE DRAWINGS

Presentations of example devices and methods:

FIG. 1 presents a stent in an expanded configuration, the stent having a reinforcement wire;

FIG. 2 presents the stent of FIG. 1 in a constrained configuration;

FIG. 3A presents a stent in an expanded configuration, the stent having a z-stent;

FIG. 3B presents the stent of FIG. 3A in a constrained configuration;

FIG. 4A presents a stent in an expanded configuration, the stent having an overlapping z-stent;

FIG. 4B presents the stent of FIG. 4A in a constrained configuration;

FIG. 5A presents a stent in an expanded configuration, the stent having a barb;

FIG. 5B presents the stent of FIG. 5A in a constrained configuration;

FIG. 6A presents a stent in an expanded configuration, the stent having a flange and a backstop;

FIG. 6B presents the stent of FIG. 6A in a constrained configuration;

FIG. 7 presents an end of an expanded stent with a flange without a backstop bending backward away from an opposite end of the tube;

FIG. 8A presents an end of an expanded stent, the stent having a flange and a backstop oriented radially outward and away from an opposite end of a tube;

FIG. 8B presents the end of FIG. 8A in a constrained configuration;

FIG. 9 presents a stent in a constrained configuration, the stent having a reinforcement wire forming a loop;

FIG. 10 presents a stent in an expanded configuration, the stent having elements from FIGS. 1, 2, 4A, 4B, 5A, 5B, 6A and 6B;

FIG. 11 presents the stent of FIG. 10 in a constrained configuration;

FIG. 12 presents a stent in an expanded configuration formed from a single continuous wire;

FIG. 13 presents a liver to be subjected to a transjugular Intrahepatic Portosystemic Shunt procedure;

FIG. 14 presents a first step in the transjugular intrahepatic portosystemic shunt procedure;

FIG. 15 presents a second step in the transjugular intrahepatic portosystemic shunt procedure;

FIG. 16 presents a third step in the transjugular intrahepatic portosystemic shunt procedure;

FIG. 17 presents a fourth step in the transjugular intrahepatic portosystemic shunt procedure;

FIG. 18 presents a fifth step in the transjugular intrahepatic portosystemic shunt procedure;

FIG. 19 presents a sixth step in the transjugular intrahepatic portosystemic shunt procedure;

FIG. 20 presents a liver after the transjugular intrahepatic portosystemic shunt procedure;

FIG. 21 presents a stent delivery device prior to stent deployment;

FIG. 22 presents the stent delivery device with a stent partly deployed; and,

FIG. 23 presents the stent delivery device after the stent of FIG. 22 is fully deployed.

### DESCRIPTION

The following description includes (1) a first section of definitions, (2) a second section with a description of selected examples of devices and methods, and (3) a third section with a more detailed description of selected examples of devices and methods.

### 1. Definitions

As used herein, the term “resilient” means that the element can be stressed with a deforming force and will spring back to its original configuration, or very close to it, when the deforming force is removed. The term “self-expanding” means the described element defines a geometric shape that encloses a volume, such as a tube, and is resilient. The term “diameter” means outside diameter. The term “attached” means connected in close proximity with or without intermediate structure between the elements. The term “contiguous” means the elements are connected and directly touching. The terms “anatomy” and “anatomical” refer to the physical structure of an organism, such as a human. The term “such as” means “for example”.

### 2. Description of Selected Examples

Various examples are presented in FIGS. 1 through 23, which are not drawn to any particular scale, and wherein like components in the numerous views are numbered alike.

### EXAMPLE 1

Referring now to FIG. 1, a first example stent 10 for placement in an anatomical passageway is provided that comprises: a self-expanding tube such as self-expanding tube 12 having an expanded tube configuration such as expanded tube configuration 60 that defines an expanded tube diameter such as expanded tube diameter 14 and a longitudinal axis such as longitudinal axis 16,
the tube comprising a first wire such as first wire 6 and at least a second wire such as second wire 8 both helically positioned along the longitudinal axis 16 forming a braid such as braid 72 at the expanded tube diameter 14,
the first wire comprising first wire portions such as first wire portions 18, the second wire comprising second wire portions such as second wire portions 20,
the first wire portions being oriented at an acute first angle such as acute first angle 22 clockwise relative to the longitudinal axis 16,
the second wire portions being oriented at an acute second angle such as acute angle 24 counter-clockwise relative to the longitudinal axis 16,
a reinforcement wire such as reinforcement wire 30 helically positioned along the longitudinal axis and adjacent the first wire portions and the second wire portions,
the reinforcement wire comprising a third wire portion such as third wire portion 36 oriented at an acute third angle such as acute third angle 32 relative to the longitudinal axis, and a fourth wire portion such as fourth wire portion 38 adjacent to the third wire portion and oriented at an acute fourth angle such as acute fourth angle 33 relative to the longitudinal axis that is in the same direction as the third angle and is less than the third angle, the third angle being greater
than the first angle and the second angle; and,

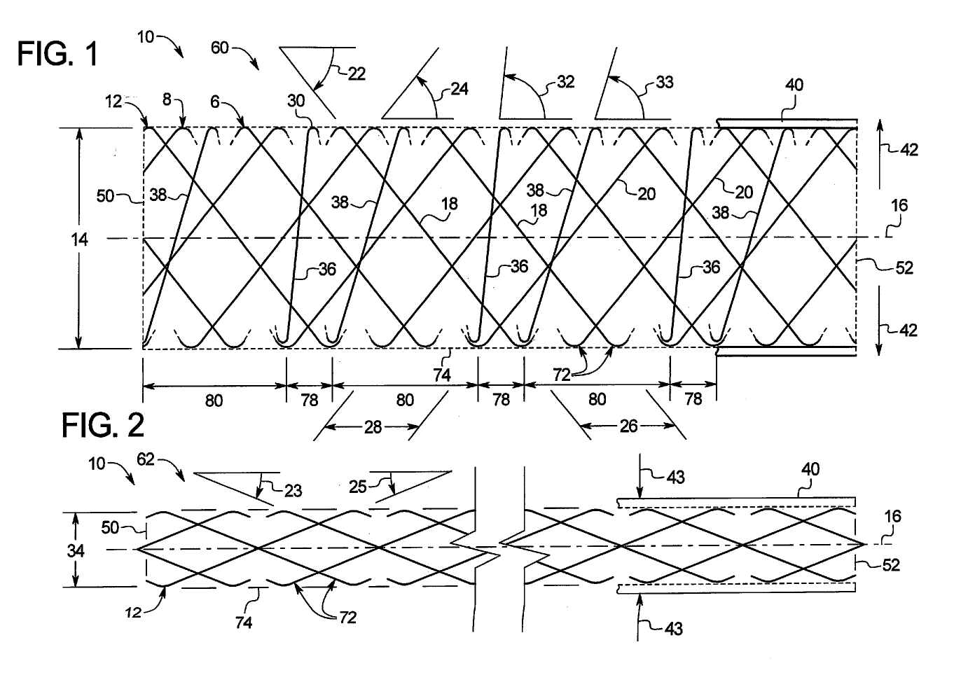

with reference to FIG. 2, the tube has a constrained tube configuration such as constrained tube configuration 62 with a decreased first angle, such as decreased first angle 23, and a decreased second angle, such as decreased second angle 25, and that defines a constrained tube diameter such as constrained tube diameter 34 for delivery to the anatomical passageway, the constrained tube diameter being less than the expanded tube diameter.

In all examples presented herein, the reinforcement wire such as reinforcement wire 30 may be woven into the braid.

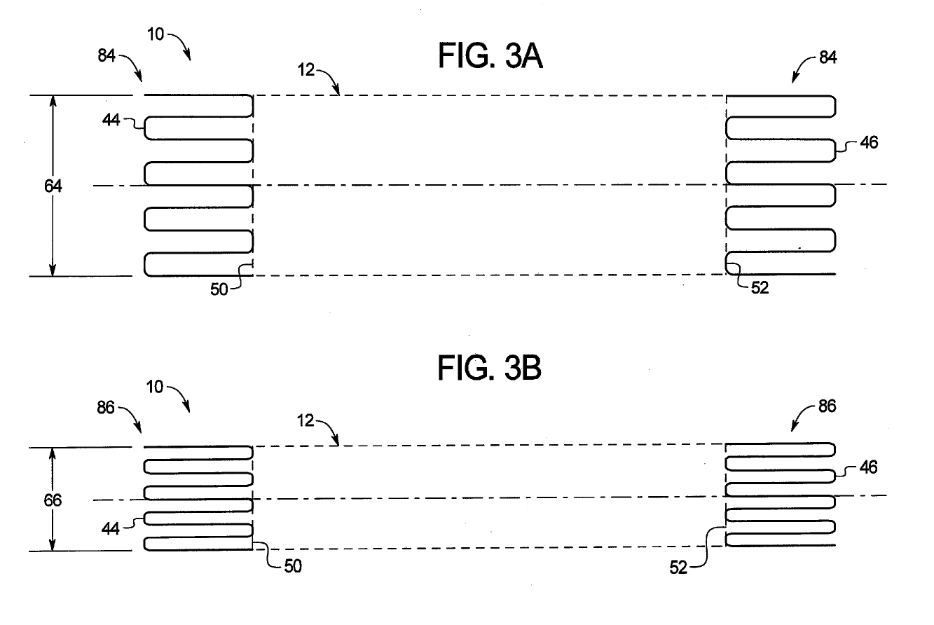

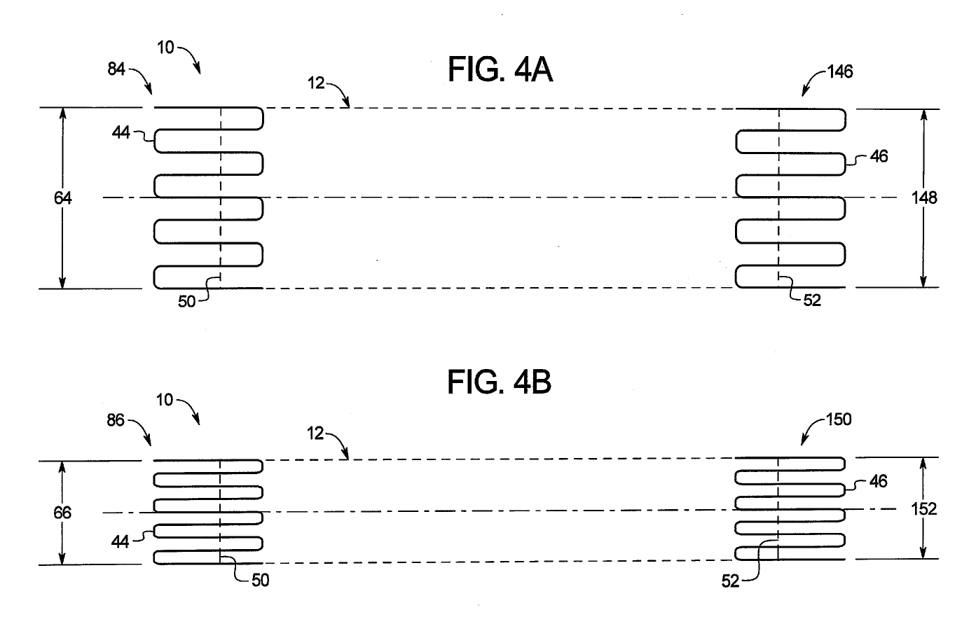

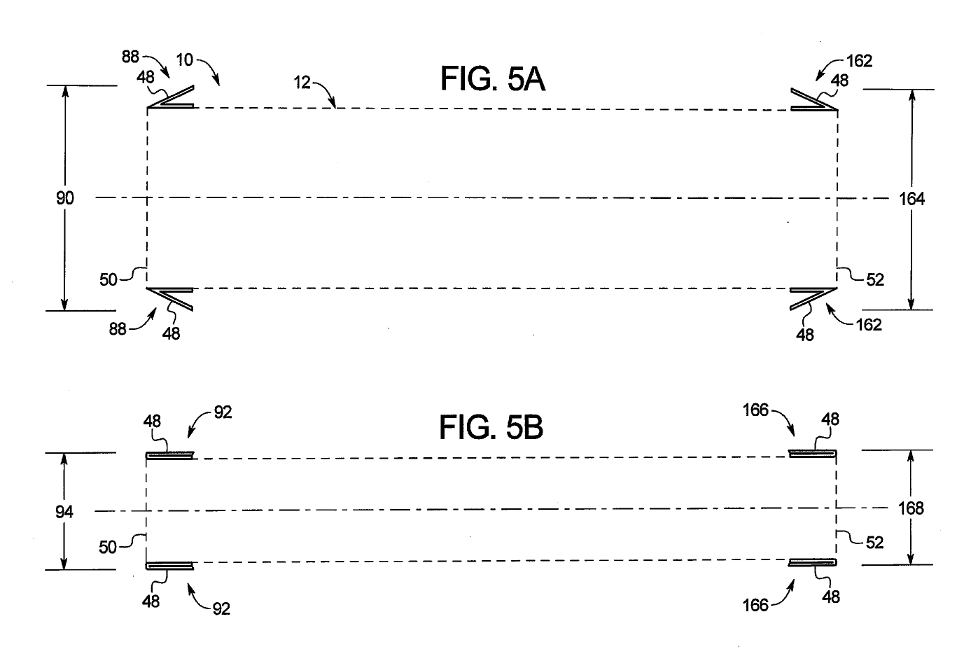

Referring now to FIGS. 3A through 4B, examples are presented wherein a first z-stent such as first z-stent 44 may be attached to an end such as end 50 of a tube such as tube 12 and a second z-stent such as second z-stent 46 may be attached to an opposite end such as opposite end 52 of the tube, the first z-stent and the second z-stent being self-expanding, the first z-stent having an expanded first z-stent configuration such as expanded first z-stent configuration 84 that defines an expanded first z-stent diameter such as expanded first z-stent diameter 64 and a constrained first z-stent configuration such as constrained first z-stent configuration 86 that defines a constrained first z-stent diameter such as constrained first z-stent diameter 66, the second -stent having an expanded second z-stent configuration such as expanded second z-stent configuration 146 that defines an expanded second z-stent diameter such as expanded second z-stent diameter 148 and a constrained second z-stent configuration such as constrained second z-stent configuration 150 that defines a constrained second z-stent diameter such as constrained second z-stent diameter 152, the constrained first z-stent diameter being less than the expanded first z-stent diameter and the constrained second z-stent diameter being less than the expanded second z-stent diameter. As specifically shown in FIGS. 4A and 4B, a first z-stent such as first z-stent 44 may overlap an end such as end 50 of the tube and a second z-stent such as second z-stent 46 may overlap an opposite end such as opposite end 52 of the tube. The z-stents that overlap the ends of tube have the same characteristics as those just described with reference to the z-stents attached to the ends of the tube. The elements of FIGS. 3A, 3B, 4A and 4B, may be implemented with the elements shown in FIGS. 5A, 5B, 6A, 6B, 8A and 8B either alternatively or in addition to one another.

Referring now to FIGS. 5A and 5B, an example is presented wherein a first barb such as first barb 48 may be attached to an end such as end 50 of the tube 12 and oriented radially outward toward an opposite end such as opposite end 52 of the tube 12, the first barb having an expanded first barb configuration such as expanded first barb configuration 88 that defines an expanded first barb diameter such as expanded first barb diameter 90 and a constrained first barb configuration such as constrained first barb configuration 92 that defines a constrained first barb diameter such as constrained first barb diameter 94, the constrained first barb diameter being less than the expanded first barb diameter. This may be implemented with the elements shown in FIGS. 3A, 3B, 4A, 4B, 6A, 6B, 8A and 8B either alternatively or in addition to one another.

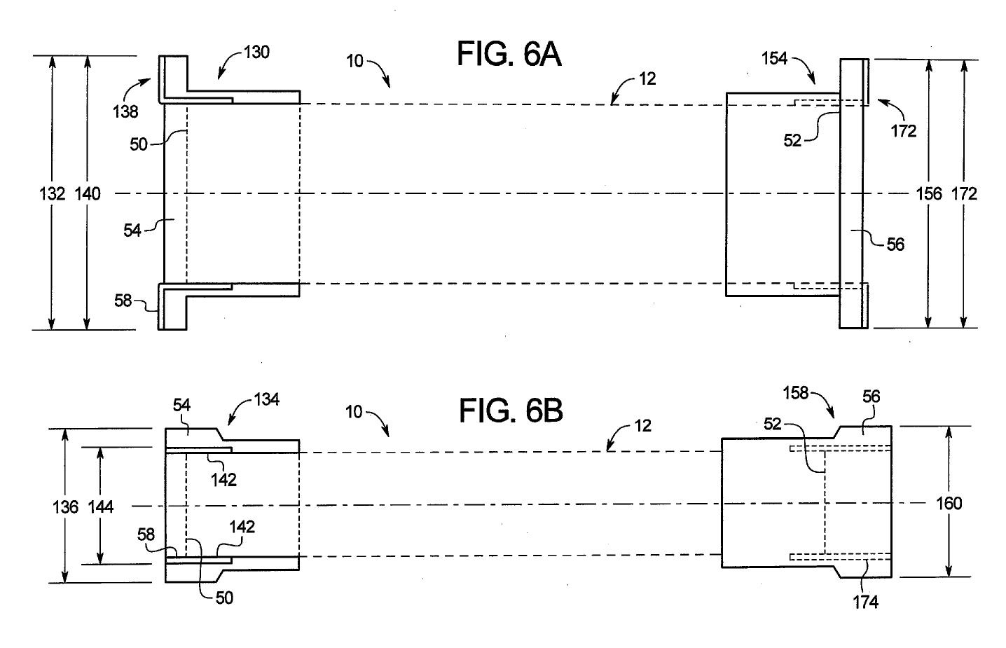

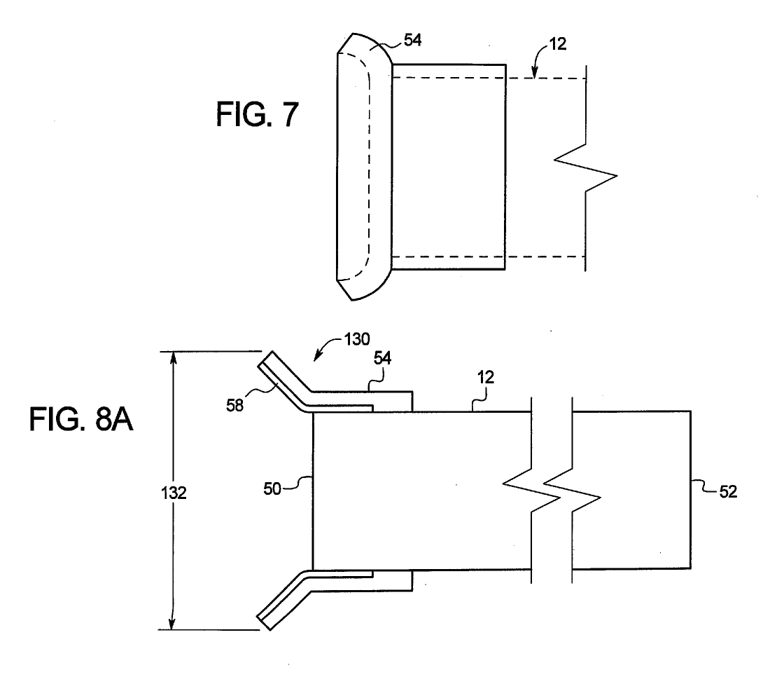

Referring now to FIGS. 6A, 6B and 7, an example is presented wherein a first flange such as first flange 54 may be attached to an end such as end 50 of a tube such as tube 12 and a first backstop such as first backstop 58 may be attached to the end of the tube and positioned to resist the first flange from bending backward away from an opposite end of the tube such as opposite end 52, as shown in FIG. 7, the first flange having an expanded first flange configuration such as expanded first flange configuration 130 that defines an expanded first flange diameter such as expanded first flange diameter 132 and a constrained first flange configuration such as constrained first flange configuration 134 that defines a constrained first flange diameter such as constrained first flange diameter 136, the constrained first flange diameter being less than the expanded first flange diameter, the first backstop having an expanded first backstop configuration such as expanded first backstop configuration 138 that defines an expanded first backstop diameter such as expanded first backstop diameter 140, and a constrained first backstop configuration such as constrained first backstop configuration 142 that defines a constrained first backstop diameter such as constrained first backstop diameter 144, the constrained first backstop diameter being less than the expanded first backstop diameter. This may be implemented with the elements shown in FIGS. 3A, 3B, 4A, 4B, 5A, 5B, 8A, and 8B either alternatively or in addition to one another.

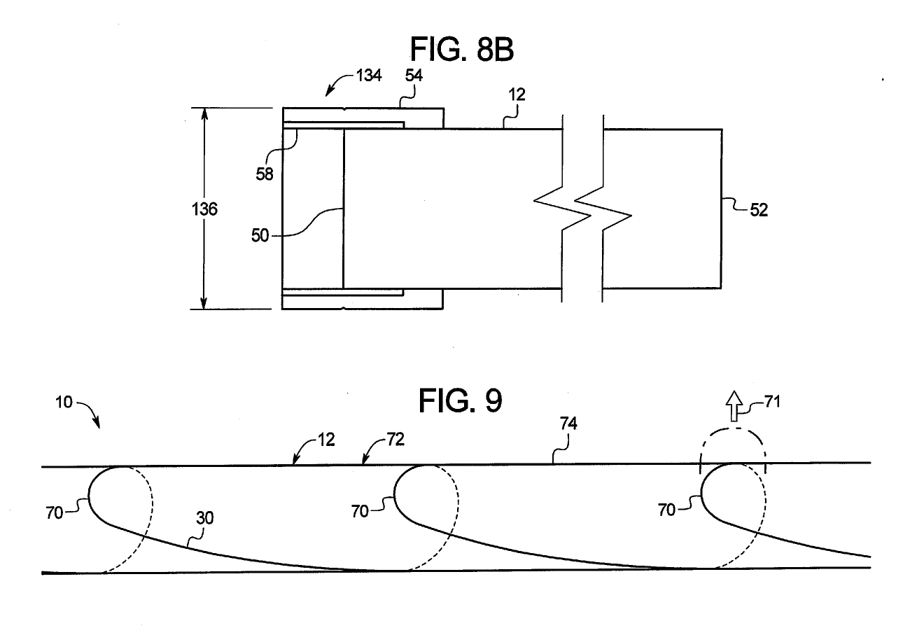

Referring now to FIGS. 8A and 8B, an example is presented wherein a first flange such as first flange 54 may be attached to an end such as end 50 of a tube such as tube 12 and a first backstop such as first backstop 58 may be attached to the end of the tube and positioned to resist the first flange 54 from bending backward away from an opposite end of the tube such as opposite end 52, the first flange and the first backstop are oriented radially outward and away from an opposite end 52 of the tube, the first flange having an expanded first flange configuration such as expanded first flange configuration 130 that defines an expanded first flange diameter such as expanded first flange diameter 132 and a constrained first flange configuration such as constrained first flange configuration 134 that defines a constrained first flange diameter such as constrained first flange diameter 136, the constrained first flange diameter being less than the expanded first flange diameter, the first backstop having an expanded first backstop configuration such as expanded first backstop configuration 138 that defines an expanded first backstop diameter such as expanded first backstop diameter 140, and a constrained first backstop configuration such as constrained first backstop configuration 142 that defines a constrained first backstop diameter such as constrained first backstop diameter 144, the constrained first backstop diameter being less than the expanded first backstop diameter. This may be implemented with the elements shown in FIGS. 3A, 3B, 4A, 4B, 5A, 5B, 6A and 6B either alternatively or in addition to one another.

### EXAMPLE 2

Referring now to FIGS. 1, 2 and 9, an example of a stent 10 having some alternate features is presented, the stent 10 being for placement in an anatomical passageway. As previously described, the stent 10 has a self-expanding tube such as self-expanding tube 12 having an expanded tube configuration such as expanded tube configuration 60 that defines a expanded tube diameter such as diameter 14 and a longitudinal axis such as longitudinal axis 16,
the tube comprising a first wire such as first wire 6 and at least a second wire such as second wire 8 both helically positioned along the longitudinal axis 16 forming a braid such as braid 72 at the expanded tube diameter,
the first wire comprising first wire portions such as first wire portions 18, the second wire comprising second wire portions such as second wire portions 20,
the first wire portions 18 being oriented at an acute first angle such as acute first angle 22 clockwise relative to the longitudinal axis,
the second wire portions being oriented at an acute second angle such as acute second angle 24 counter-clockwise relative to the longitudinal axis,
a reinforcement wire such as reinforcement wire 30 helically positioned along the longitudinal axis and adjacent the first wire portions and the second wire portions,
at least a portion of the reinforcement wire being oriented at an acute third angle such as acute third angle 32 relative to the longitudinal axis,
the third angle being greater than the first angle and the second angle; and,

with reference to FIGS. 2 and 9, the tube has a constrained tube configuration such as constrained tube configuration 62 with a decreased first angle such as decreased first angle 23 and a decreased second angle such as decreased second angle 25 and that defines a constrained tube diameter such as constrained tube diameter 34 for delivery to the anatomical passageway, the constrained tube diameter being less than the expanded tube diameter, and, as shown in FIG. 9, the reinforcement wire forming a loop, such as loop 70, in the constrained tube configuration that does not exist in the expanded tube configuration.

Referring again to FIGS. 1, 2, and 9, an example is presented wherein a braid such as braid 72 may define a wall such as wall 74, the loop such as loop 70 extending within the wall.

Still referring to FIGS. 1, 2, and 9, an example is presented wherein a braid such as braid 72 may define a wall such as wall 74, the loop such as loop 70 extending outside the wall, as indicated by arrow 71. This may be implemented with the loop extending within the wall. There may be a combination of loops extending within the wall 74 and outside the wall 74. This may be implemented with the elements of FIGS. 3A, 3B, 4A, 4B, 5A, 5B, 6A, 6B, 8A and 8B in the alternative or in addition to one another.

As previously described in Example 1 with reference to FIG. 1, a reinforcement wire such reinforcement wire 30 may comprise a third wire portion such as third wire portion 36 oriented at an acute third angle such as acute third angle 32 relative to the longitudinal axis, and a fourth wire portion such as fourth wire portion 38 adjacent to the third wire portion and oriented at an acute fourth angle such as acute fourth angle 33 relative to the longitudinal axis that is in the same direction as the third angle and is less than the third angle, the third angle being greater than the first angle and the second angle. This may be implemented with the elements of FIGS. 3A, 3B, 4A, 4B, 5A, 5B, 6A, 6B, 8A, and 8B in the alternative or in addition to one another.

As previously described in Example 1 with reference to FIGS. 3A, 3B, 4A and 4B, examples are presented wherein a first z-stent such as first z-stent 44 may be attached to an end such as end 50 of a tube such as tube 12 and a second z-stent such as second z-stent 46 may be attached to an opposite end such as opposite end 52 of the tube, the first z-stent and the second z-stent being self-expanding, the first z-stent having an expanded first z-stent configuration such as expanded first z-stent configuration 84 that defines an expanded first z-stent diameter such as expanded first z-stent diameter 64 and a constrained first z-stent configuration such as constrained first z-stent configuration 86 that defines a constrained first z-stent diameter such as constrained first z-stent diameter 66, the second z-stent having an expanded second z-stent configuration such as expanded second z-stent configuration 146 that defines an expanded second z-stent diameter such as expanded second z-stent diameter 148 and a constrained second z-stent configuration such as constrained second z-stent configuration 150 that defines a constrained second z-stent diameter such as constrained second z-stent diameter 152, the constrained first z-stent diameter being less than the expanded first z-stent diameter and the constrained second z-stent diameter being less than the expanded second z-stent diameter. As specifically shown in FIGS. 4A and 4B, a first z-stent such as first z-stent 44 may overlap an end such as end 50 of the tube and a second z-stent such as second z-stent 46 may overlap an opposite end such as opposite end 52 of the tube. The z-stents that overlap the ends of tube have the same characteristics as those just described with reference to the z-stents attached to the ends of the tube. The examples of FIGS. 3A, 3B, 4A and 4B, may be implemented with the elements shown in FIGS. 5A, 5B, 6A, 6B, 8A and 8B either alternatively or in addition to one another.

As previously described in Example 1 with reference to FIGS. 5A and 5B, an example is presented wherein a first barb such as barb 48 may be attached to an end such as end 50 of the a tube such as tube 12 and oriented radially outward toward an opposite end such as opposite end 52 of the tube, the first barb having an expanded first barb configuration such as expanded first barb configuration 88 that defines an expanded first barb diameter such as expanded first barb diameter 90 and a constrained first barb configuration such as constrained first barb configuration 92 that defines a constrained first barb diameter such as constrained first barb diameter 94, the constrained first barb diameter being less than the expanded first barb diameter. This may be implemented with the elements shown in FIGS. 3A, 3B, 4A, 4B, 6A, 6B, 8A and 8B either alternatively or in addition to one another.

As previously described in Example 1 with reference to FIGS. 6A, 6B and 7, an example is presented wherein a first flange such as first flange 54 may be attached to an end such as end 50 of a tube such as tube 12 and a first backstop such as first backstop 58 may be attached to the end of the tube and positioned to resist the first flange from bending backward away from an opposite end of the tube such as opposite end 52, as shown in FIG. 7, the first flange having an expanded first flange configuration such as expanded first flange configuration 130 that defines an expanded first flange diameter such as expanded first flange diameter 132 and a constrained first flange configuration such as constrained first flange configuration 134 that defines a constrained first flange diameter such as constrained first flange diameter 136, the constrained first flange diameter being less than the expanded first flange diameter, the first backstop having an expanded first backstop configuration such as expanded first backstop configuration 138 that defines an expanded first backstop diameter such as expanded first backstop diameter 140, and a constrained first backstop configuration such as constrained first backstop configuration 142 that defines a constrained first backstop diameter such as constrained first backstop diameter 144, the constrained first backstop diameter being less than the expanded first backstop diameter. This may be implemented with the elements shown in FIGS. 3A, 3B, 4A, 4B, 5A, 5B, 8A, and 8B either alternatively or in addition to one another.

As previously described in Example 1 with reference to FIGS. 8A and 8B, an example is presented wherein a first flange such as first flange 54 may be attached to an end such as end 50 of a tube such as tube 12 and a first backstop such as backstop 58 may be attached to the end of the tube and positioned to resist a first flange such as first flange 54 from bending backward away from an opposite end of the tube such as opposite end 52, the first flange and the first backstop are oriented radially outward and away from an opposite end of the tube the first flange having an expanded first flange configuration 130 that defines an expanded first flange diameter such as expanded first flange diameter 132 and a constrained first flange configuration such as constrained first flange configuration 134 that defines a constrained first flange diameter such as constrained first flange diameter 136, the constrained first flange diameter being less than the expanded first flange diameter, the first backstop having an expanded first backstop configuration such as expanded first backstop configuration 138 that defines an expanded first backstop diameter such as expanded first backstop diameter 140, and a constrained first backstop configuration such as constrained first backstop configuration 142 that defines a constrained first backstop diameter such as constrained first backstop diameter 144, the constrained first backstop diameter being less than the expanded first backstop diameter. This may be implemented with the elements shown in FIGS. 3A, 3B, 4A, 4B, 5A, 5B, 6A and 6B either alternatively or in addition to one another.

### EXAMPLE 3

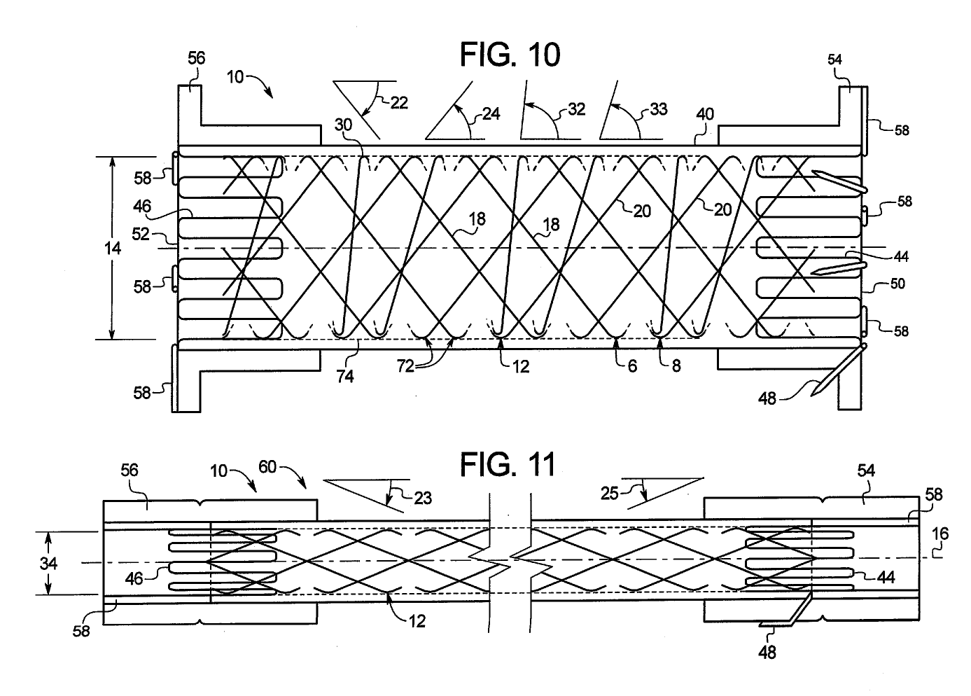

Referring now to FIG. 10, a third example of a stent 10 for placement in an anatomical passageway is presented, which comprises:

a self-expanding tube such as self-expanding tube 12 having an expanded tube configuration such as expanded tube configuration 60 that defines an expanded tube diameter such as expanded tube diameter 14 and a longitudinal axis such as longitudinal axis 16,
the tube comprising a first wire such as first wire 6 and at least a second wire such as second wire 8 both helically positioned along the longitudinal axis forming a braid such as braid 72 at the expanded tube diameter,
the first wire comprising first wire portions such as first wire portions 18, the second wire comprising second wire portions such as second wire portions 20,
the first wire portions being oriented at an acute first angle such as acute first angle 22 clockwise relative to the longitudinal axis,
the second wire portions being oriented at an acute second angle such as acute second angle 24 counter-clockwise relative to the longitudinal axis,
a reinforcement wire such as reinforcement wire 30 helically positioned along the longitudinal axis and adjacent the first wire portions and the second wire portions,
the reinforcement wire comprising a third wire portion such as third wire portion 36 oriented at an acute third angle such as acute third angle 32 relative to the longitudinal axis, and a fourth wire portion such as fourth wire portion 38 adjacent to the third wire portion and oriented at an acute fourth angle such as acute fourth angle 33 relative to the longitudinal axis that is in the same direction as the third angle and is less than the third angle, the third angle being greater than the first angle and the second angle; and,

with reference to FIG. 11, the tube such as tube 12 has a constrained configuration such as constrained tube configuration 62 with a decreased first angle such as decreased first angle 23 and a decreased second angle such as decreased second angle 25 and that defines a constrained tube diameter such as constrained tube diameter 34 for delivery to the anatomical passageway, the constrained tube diameter being less than the expanded tube diameter; and,

a first z-stent such as first z-stent 44 overlapping an end such as end 50 of the tube 12, first z-stent 44 being self-expanding and contiguous with the tube, the first z-stent having an expanded first z-stent configuration such as expanded first z-stent configuration 84 that defines an expanded first z-stent diameter such as expanded first z-stent diameter 64 and a constrained first z-stent configuration such as constrained first z-stent configuration 86 that defines a constrained first z-stent diameter such as constrained first z-stent diameter 66, the constrained first z-stent diameter being less than the expanded first z-stent diameter;

a second z-stent such as second z-stent 46 overlapping an opposite end such as opposite end 52 of the tube, the second z-stent being self-expanding and contiguous with the tube, the second z-stent having an expanded second z-stent configuration such as expanded second z-stent configuration 146 that defines an expanded second z-stent diameter such as expanded second z-stent diameter 148 and a constrained second z-stent configuration such as constrained second z-stent configuration 150 that defines a constrained second z-stent diameter such as constrained second z-stent diameter 152, the constrained second z-stent diameter being less than the expanded second z-stent diameter;

a first flange such as first flange 54 contiguous with the first z-stent, the first flange having an expanded first flange configuration such as expanded first flange configuration 130 that defines an expanded first flange diameter such as expanded first flange diameter 132 and a constrained first flange configuration such as constrained first flange configuration 134 that defines a constrained first flange diameter such as constrained first flange diameter 136, the constrained first flange diameter being less than the expanded first flange diameter;

a second flange such as second flange 56 contiguous with the second z-stent, the second flange having an expanded second flange configuration such as expanded second flange configuration 154 that defines an expanded second flange diameter such as expanded second flange diameter 156 and a constrained second flange configuration such as constrained second flange configuration 158 that defines a constrained second flange diameter such as second flange diameter 160, the constrained second flange diameter being less than the expanded second flange diameter;

a first backstop such as backstop 58 contiguous with the first z-stent positioned to resist the first flange from bending backward away from the opposite end of the tube, the first backstop having an expanded first backstop configuration such as expanded first backstop configuration 138 that defines an expanded first backstop diameter such as expanded first backstop diameter 140, and a constrained first backstop configuration such as constrained first backstop configuration 142 that defines a constrained first backstop diameter such as constrained first backstop diameter 144, the constrained first backstop diameter being less than the expanded first backstop diameter;

a second backstop such as backstop 59 contiguous with the second z-stent positioned to resist the second flange from bending backward away from the end of the tube, the second backstop having an expanded second backstop configuration such as expanded second backstop configuration 170 that defines an expanded second backstop diameter such as expanded second backstop diameter 172, and a constrained second backstop configuration such as constrained second backstop configuration 174 that defines a constrained second backstop diameter such as constrained second backstop diameter 176, the constrained second backstop diameter being less than the expanded second backstop diameter;

a first barb such as barb 48 contiguous with the first z-stent and oriented radially outward toward the opposite end of the tube, the barb having an expanded first barb configuration such as expanded first barb configuration 88 that defines an expanded first barb diameter such as expanded first barb diameter 90 and a constrained first barb configuration such as constrained first barb configuration 92 that defines a constrained first barb diameter such as constrained first barb diameter 94, the constrained first barb diameter being less than the expanded first barb diameter; and,

a constrainable covering layer such as constrainable covering layer 40 contiguous with the tube that covers at least the tube that is not covered by the first flange and the second flange.

### EXAMPLE 4

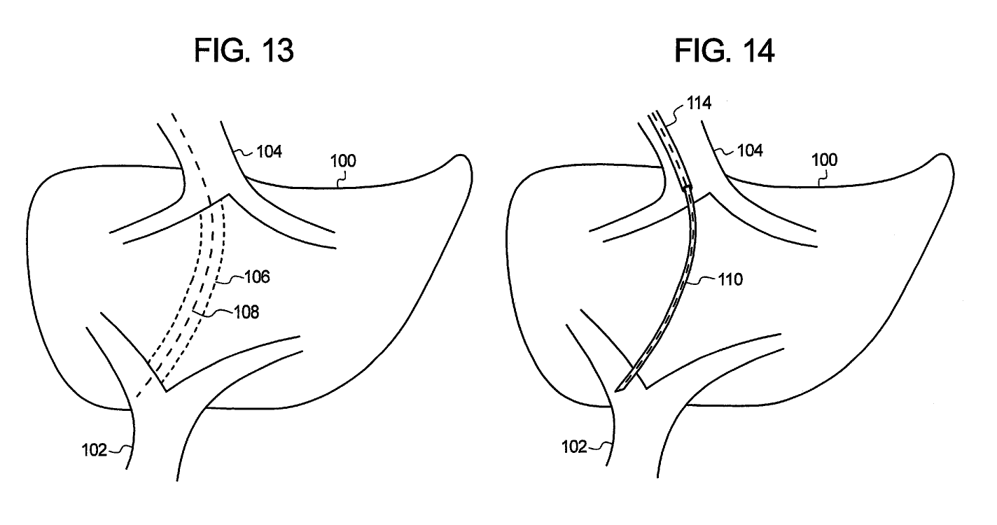

Referring now to FIGS. 13 to 20, an example of a method of placing a stent in a liver 100 between a portal vein 102 and a hepatic vein 104 is presented, Examples 1, 2, or 3 being examples of stents that can be used, but not limited to these examples, comprising:

percutaneously accessing the hepatic vein 104;

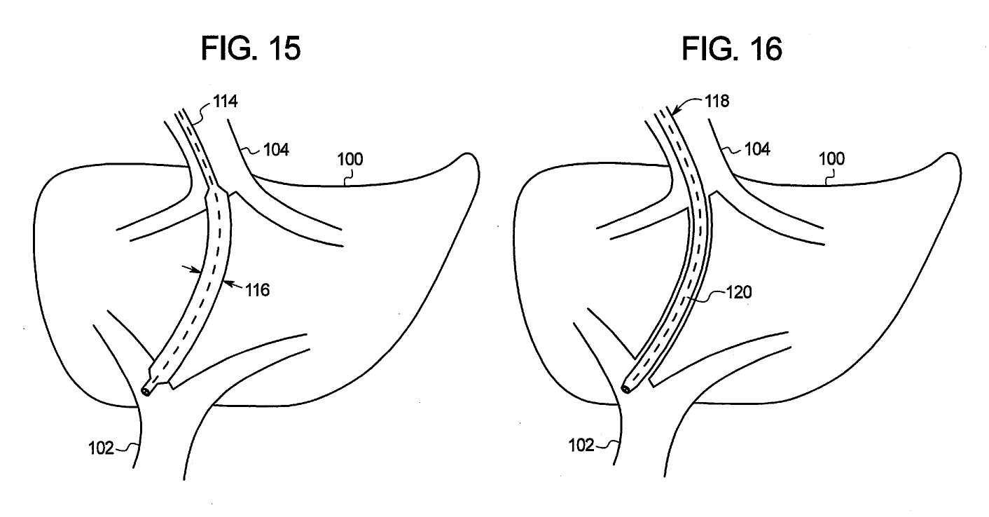

as shown in FIGS. 14 and 15, forming a passageway 106 in the liver 100 between the portal vein 102 and the hepatic vein 104;

as shown in FIG. 16, placing a self-expanding tube such as tube 12 in the passageway 106,
the self-expanding tube having an expanded tube configuration such as expanded tube configuration 60 that defines an expanded tube diameter such as expanded tube diameter 14 and a longitudinal axis such as longitudinal axis 16,
   the tube comprising a first wire such as first wire 6 and at least a second wire such as second wire 8 both helically positioned along the longitudinal axis forming a braid such as braid 72 at the expanded tube diameter,
   the first wire comprising first wire portions such as first wire portions 18, the second wire comprising second wire portions such as second wire portions 20,
   the first wire portions being Oriented at an acute first angle such as acute angle 22 clockwise relative to the longitudinal axis,
   the second wire portions being oriented at an acute second angle such as acute second angle 24 counter-clockwise relative to the longitudinal axis,
   a reinforcement wire such as reinforcement wire 30 helically positioned along the longitudinal axis and adjacent the first wire portions and the second wire portions,
   the reinforcement wire comprising a third wire portion such as third wire portion 36 oriented at an acute third angle such as acute third angle 32 relative to the longitudinal axis, and at least least one fourth wire portion such as fourth wire portion 38 adjacent to the third wire portions and oriented at an acute fourth angle such as acute fourth angle 33 relative to the longitudinal axis that is in the same direction as the third angle and is less than the third angle, the third angle being greater than the first angle and the second angle; and,
with reference to FIG. 2, the tube has a constrained tube configuration such as constrained tube configuration 62 with a decreased first angle such as decreased first angle 23 and a decreased second angle such as decreased second angle 25 and that defines a constrained tube diameter such as constrained tube diameter 34 for delivery to the anatomical passageway 106, the constrained tube diameter being less than the expanded tube diameter, the tube 12 being in the constrained tube configuration within a stent sheath such as stent sheath 120; and,

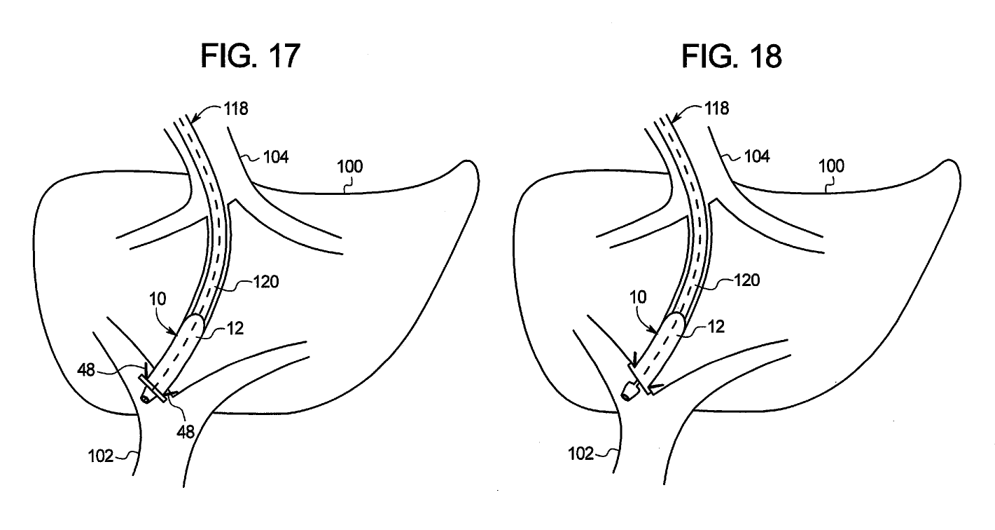

expanding from the constrained tube configuration to the expanded tube configuration by removing the stent sheath from the tube, and leaving the tube in the passageway 106, as shown in FIGS. 17 through 20.

In this example of a method, the device may comprise a first barb such as first barb 48 attached to an end such as end 50 of the tube and oriented radially outward toward an opposite end of the tube such as opposite end 52, the first barb having an expanded first barb configuration such as expanded first barb configuration 88 that defines an expanded first barb diameter such as expanded first barb diameter 90 and a constrained first barb configuration such as constrained first barb configuration 92 that defines a constrained first barb diameter such as constrained first barb diameter 94, the constrained first barb diameter being less than the expanded first barb diameter, the first barb being in the constrained configuration within the stent sheath,

the method comprising:

partially expanding the end of the tube and the first barb into the portal 102 vein so the tube is partially in the expanded tube configuration and the first barb is in the expanded first barb configuration by partially removing the stent sheath from the tube, as shown in FIG. 17;

pulling the tube toward the hepatic vein 104 to implant the first barb in the portal vein 102 and to stretch the tube, as shown in FIG. 18; and,

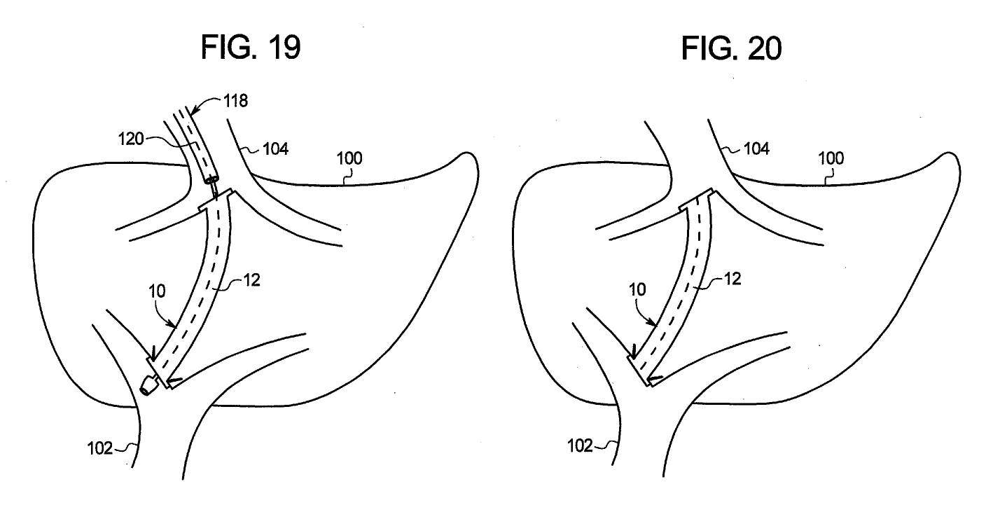

expanding the rest of the tube within the passageway, as shown in FIG. 19 so the tube is in the expanded stent configuration by completely removing the stent sheath from the tube.

In this example of a method,
the tube may comprise a first barb such as barb 48 attached to an end such as end 50 of the tube and oriented radially outward toward an opposite end such as opposite end 52 of the tube, the first barb having an expanded first barb configuration such as expanded first barb configuration 88 that defines an expanded first barb diameter such as expanded first barb diameter 90 and a constrained first barb configuration such as constrained first barb configuration 92 that defines a constrained first barb diameter such as constrained first barb diameter 94, the constrained first barb diameter being less than the expanded first barb diameter, the first barb being in the constrained first barb configuration within the stent sheath, and
a first flange such as first flange 54 attached to an end such as end 50 of the tube and a second flange such as second flange 56 attached to an opposite end such as opposite end 52 of the tube, the first flange having an expanded first flange configuration such as expanded first flange configuration 130 that defines an expanded first flange diameter such as expanded first flange diameter 132 and the first flange having a constrained first flange configuration such as constrained first flange configuration 134 that defines a constrained first flange diameter such as constrained first flange diameter 136, the constrained first flange diameter being less than the expanded first flange diameter, the first flange being in the constrained first flange configuration within the stent sheath, and
the second flange having an expanded second flange configuration such as expanded second flange configuration 154 that defines an expanded second flange diameter such as expanded second flange diameter 156 and a constrained second flange configuration such as constrained second flange configuration 158 that defines a constrained second flange diameter such as constrained second flange diameter 160, the constrained second flange diameter being less than the expanded second flange diameter, the second flange being in the constrained second flange configuration within the stent sheath, and
a constrainable covering layer such as constrainable covering layer 40 attached to the tube that covers at least the tube that is not covered by the first flange and the second flange, the constrainable covering layer being constrained with the tube, the method comprising:

partially expanding the end of the tube and fully expanding the first barb and the first flange into the portal vein 102 by partially removing the stent sheath from the tube, as shown in FIG. 17;

pulling the tube toward the hepatic vein 104 to implant the barb in the portal vein 102 and to stretch the tube, as shown in FIG. 18; and,

expanding the rest of the tube with the second flange in the hepatic vein 104, as shown in FIG. 19, by completely removing the stent sheath from the tube.

In this example of a method, the tube may comprise a first flange such as first flange 54 attached to an end of the tube and a first backstop such as first backstop 58 attached to the end of the tube and positioned to resist the first flange from bending backward away from an opposite end of the tube such as opposite end 52, the first flange and the first backstop oriented radially outward and away from an opposite end such of the tube, the first flange having an expanded first flange configuration such as expanded first flange configuration 130 that defines an expanded first flange diameter such as expanded first flange diameter 132 and a constrained first flange configuration such as . constrained first flange configuration 134 that defines a constrained first flange diameter such as constrained first flange diameter 136, the constrained first flange diameter being less than the expanded first flange diameter, the first flange being in the constrained first flange configuration, the first backstop having an expanded first backstop configuration such as expanded first backstop configuration 138 that defines an expanded first backstop diameter such as expanded first backstop diameter 140, and a constrained first backstop configuration such as constrained first backstop configuration 142 that defines a constrained first backstop diameter such as constrained first backstop diameter 144, the constrained first backstop diameter being less than the expanded first backstop diameter, the first flange being in the constrained first flange configuration within the stent sheath and the first backstop being in the constrained first backstop configuration within the stent sheath prior to the step of expanding from the constrained tube configuration to the expanded tube configuration by removing the stent sheath from the tube.

### 3. Detailed Description of Selected Examples

Referring again to FIG. 1, and as mentioned previously, self-expanding means that the tube 12 is resilient. It can be radially deformed with a constraining force and energy is stored as elastic deformation energy in the tube 12. The tube 12 springs back to its original shape, or very close to it, when the constraining force, a radial inward force 43, is removed. The first wire 6 and second wire 8 may be formed from a resilient material, as well as the reinforcement wire 30, meaning that the wire portions can be stressed with a deforming force and will spring back to their original configuration, or very close to it, when the deforming force is removed. The wires and other elements disclosed herein may be formed from a superelastic material such as nitinol or other materials having suitable elasticity to render the tube 12 self-expanding. Examples of other materials include Elgiloy or titanium without limitation. The first wire 6 and second wire 8 impart the self-expanding property to the tube 12. If a superelastic material is used in any embodiment, it may be superelastic at body temperature.

The reinforcement wire 30 may also be formed from a resilient material. The reinforcement wire applies a radial outward force 42 against the first wire 6 and the second wire 8, which adds to the self-expanding characteristic of the tube 12. The reinforcement wire 30 may be formed from a superelastic material such as nitinol or other materials having suitable elasticity to render the tube 12 self-expanding. The first wire 6, second wire 8, and reinforcement wire 30, may be formed from wire having a diameter of 0.015 (0.381 mm) inch or less. This is not a limitation as other diameters may be useful.

A braid means that the first wire 6 and second wire 8 pass over and under each other as they helically advance along the longitudinal axis 16. With a braid 72, the first angle 22 and second angle 24 may be substantially equal. The tube 12 may be braided by hand or by a machine. It may be circular in cross section perpendicular to the longitudinal axis 16. There are first spaces 26 between adjacent wire revolutions of the first wire 6, and there are second spaces 28 between adjacent wire revolutions of the second wire 8.

The braid 72 is shown schematically for the sake of clarity. In an actual braid the first wire portions 18 and second wire portions 20 appear as a soft “S” shape when the tube 12 is viewed from the side. The first wire 6, second wire 8, and reinforcement wire 30 on the backside are not shown for the sake of clarity. The first angle 22 and second angle 24 are measured where the first wire portions 18 and second wire portions 20, respectively, cross the longitudinal axis 16. The same is true of the decreased first angle 23 and decreased second angle 25. This also true of the third wire portions 36 and fourth wire portions 38 of the reinforcement wire 30. They appear as a soft “S” shape when the tube 12 is viewed from the side. The third angle 32 and fourth angle 33 are measured where the third wire portions 36 and fourth wire portions 38, respectively, cross the longitudinal axis 16.

The third wire portions 36 and fourth wire portions 38 of the reinforcement wire 30 may extend a full 360 degrees around the circumference of the tube 12 (the tube being viewed in a cross-section perpendicular to the longitudinal axis 16). Angles less than a full 360 degrees and angles greater than a full 360 degrees are contemplated.

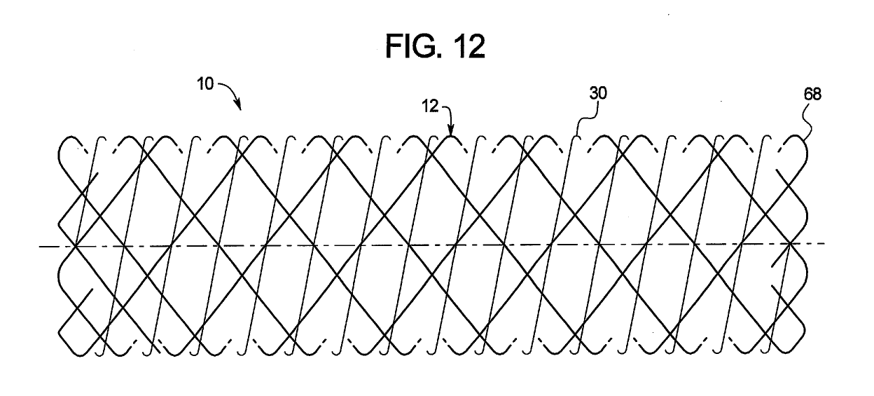

With a braid 72, the first spaces 26 and second spaces 28 may be substantially equal. The first wire 6 and second wire 8 may form a single continuous wire 31, as shown in FIG. 12. There may be a plurality of individual wires, or a multitude of individual wires.

The tube 12 may vary in cross-section along the longitudinal axis. For example it may have a linear taper (frustoconical) or it may have a maximum diameter in the middle or a minimum diameter in the middle. Potential shapes are too numerous to list and evident from the description provided herein. In this example, the first angle 22, second angle 24, first spaces 26 and second spaces 28 may vary along the longitudinal axis 16, as can the third angle 32 and fourth angle 33.

Still referring to FIG. 1, the reinforcement wire 30 may comprise a third wire portion 36 oriented at an acute third angle 32 relative to the longitudinal axis 16, and a fourth wire portion 38 adjacent to the third wire portion 36 and oriented at an acute fourth angle 33 relative to the longitudinal axis 16 that is in the same direction as the third angle 32 and is less than the third angle 32, the third angle 32 being greater than the first angle 22 and the second angle 24. The term “same direction” means that the third angle 32 and fourth angle 33 are both clockwise or counter-clockwise relative to the longitudinal axis 16 such that the third wire portions 36 and and fourth wire portions 38 are both oriented clockwise or counter-clockwise, respectively. In FIG. 1, both are oriented counter-clockwise. There may be a multitude of third wire portions 36 and fourth wire portions portions 38.

As shown in FIG. 1, the difference in third angle 32 and fourth angle 33, as they repeat in a series, generates a reinforcement wire 30 with a varying pitch. Pitch is the distance between adjacent portions of the reinforcement wire 30. For example first pitch 78 and second pitch 80. A plurality of first pitches 78 may be interleaved with a plurality of second pitches 80. This is a direct result of the third wire portion 36 being adjacent the fourth wire portion 38 and oriented at the third angle 32 and fourth angle 33, respectively, which may be repeated in a series.

The reinforcement wire 30 gives the tube 12 desirable characteristics. By nature braids tend to radially contract and longitudinally extend with relatively low force. The force can be applied as longitudinal compression or longitudinal stretching. Force can also be applied as radial compression. The resistance to radial compression of tube 12 varies with position along the longitudinal axis 16. It is greatest near the third wire portions 36 that are oriented at the acute third angle 32 because they are more vertical in orientation relative to the first angle 22 and the second angle 24 of the first wire portion portions 18 and second wire portions 20, respectively. It is least at a position between two third wire portions 36, for example. Furthermore, overall resistance to radial compression is greater than the braid 72 alone. The reinforcement wire 30 acts as a buttress.

Resistance to longitudinal stretching also increases compared to an unreinforced braid. This is because stretching the braid 72 causes it to contract. The reinforcement wire 30 resists the contraction because it acts as a buttress, as just described.

At the same time, some stretchiness is desirable because it allows the tube 12 to adjust to changes in length in an anatomical passageway. The tube 12 maintains a level of stretchiness because the braid 72 between third wire portions 36 has room to stretch compared to locations that are in close proximity to the third wire portions 36.

In addition, the third angle 32 and fourth angle 33 may change along the longitudinal axis 16. For example, the third angle 32 could have one value at one position along the longitudinal axis 16, and another value at another position along the longitudinal axis 16. Likewise, the fourth angle 32 could have one value at one position along the longitudinal axis 16, and another value at another position along the longitudinal axis 16. Also, the distance between third portions 36 may change with position along the longitudinal axis 16. This permits a selection of stent portions having greater or lesser resistance to radial compression dependent upon longitudinal position, and a selection of stent portions having greater or less stretchiness, also dependent on longitudinal position. For example, the portions with greater resistance to radial compression could be positioned to align with areas of greater radial compressive force in the anatomical passageway.

The constrained tube configuration 62 shown in FIG. 2 is generally held in place by a stent sheath 120, as are the other examples described herein, the stent sheath 120 and its operation being described later with reference to FIGS. 16 through 20, and 21 through 23. In the constrained configuration the tube may have a decreased first angle 23 and a decreased second angle 25, as shown in FIG. 2. This may occur because the third wire portions 18 and second wire portions 20 are not fixed to one another and may slide past one another and/or engage in a scissoring motion. The length of the tube 12 may increase, which is why the middle portion is shown broken in FIG. 2 purely for explanatory purposes.

As can be seen in FIG. 2, the constraining force is a radial inward force 43 (radial compression) that reduces the diameter of the stent 10 (and tube 12) and the constrained stent 10 may be held in this state by the stent sheath 120 that extends along the longitudinal axis 16. The inside diameter of the stent sheath 120 approximates the outside diameter of the constrained stent 10.

As shown in FIG. 1 the stent 10 may comprise a constrainable covering layer 40 attached to the tube 12 that covers the braid 72. The constrainable covering layer 40, first flange 54, and second flange 56 may be formed from a silicone elastomer or materials having similar properties for use as a constrainable covering layer or flange. Other materials include expanded polytetrafluoroethylene, and ultra high molecular weight polyethylene, but not limited to these materials. The constrainable layer may be partially or fully impervious to blood and fluids, for example fluids within the liver. Either all or part of the first spaces 26 and second spaces 28 may be covered. In these examples the flanges 54 and 56 are formed of materials that permit the flanges to expand upon relief from a constraining force imposed by a constrained tube configuration 62.

The reinforcement wire 30 may be woven into the braid 72 comprising the first wire 6 and second wire 8. This may be done by hand or machine during or after formation of the braid 72. When woven the reinforcement wire 30 passes over and under the first wire 6 and the second wire wire 8. The woven reinforcement wire 30 may be formed into a helical cylindrical configuration prior to placement.

Referring now to FIGS. 3A, 3B, 4A and 4B, the first z-stent 44 and the second z-stent 46 may be self-expanding, as described earlier with respect to the tube 12. They may be formed from a superelastic material such as nitinol or another material with suitable elasticity to render the tube 12 self-expanding. They are referred to as z-stents because they are formed from wires or ribbons that pass back and forth along the longitudinal axis thereby forming structures similar in shape to the letter “Z”. The first z-stent 44 and second z-stent 46 may be contiguous with the tube 12. They may be placed inside or outside of it. They may be made connected and touching the tube 12 by welding or soldering them to the tube 12 but not limited to these techniques. The expanded first z-stent diameter 64 may be lesser or greater than the expanded second z-stent diameter 148. Likewise, the constrained first z-stent diameter 66 may be lesser or greater than the constrained second z-stent diameter 152.

Referring to FIGS. 5A and 5B, a barb 48 is shown on both ends 50 and 52. However, a barb or barbs may be provided on only one end 50 or 52. There may be multiple barbs 48 on either one or both of end 50 and 52. A barb 48 or barbs 48 may be provided on opposite end 52, and this element may be implemented alone or in combination with the elements of FIGS. 1 through 4B, 6A through 8B, 10 and 11. The barb 48 may be formed from the same material as the part it is fixed to. For example, it may be nitinol and the part may be nitinol. They may also be different materials. Regardless of the materials it may be fixed by soldering or welding but not limited to these techniques. Similarly for the backstop 58, it may be formed from the same material as the part it is fixed to. For example, it may be nitinol and the part may be nitinol. They may also be different materials. Regardless of the materials it may be fixed by soldering or welding but not limited to these techniques. The barb 48 may be a separate piece and attached by welding, soldering, or other attachment technique. It may also be integral with the stent 44, 46. An example of integral is laser cutting a nitinol tube to form the stent 44, 46. The barb 48 could be formed as a part of the process. The same applies to the backstop 58. It may be formed as a part of the process of laser cutting a nitinol tube to form a stent 44, 46.

To the extent that barbs 48 are placed on the end 50, and opposite end 52, with reference to FIGS. 5A and 5B, the expanded first barb diameter 90 may be lesser or greater than an expanded second barb diameter 164. Likewise, the constrained first barb diameter 94 may be lesser or greater than the constrained second barb diameter 168. A second barb 48 attached to the opposite end 52 may have an expanded second barb configuration 162 and a constrained second barb configuration 166.

FIGS. 6A and 6B present first flange 54 and second flange 56. Flanges 54 and 56 are shown on both ends 50 and 52. However, a flange may be provided on only one end 50 or 52. The first flange 54 and second flange 56 may extend 360 degrees around the longitudinal axis 16 and, thus, fully encompass the ends 50 and 52. The flanges 54 and 56 hold the stent 10 in position within an anatomical passageway. A force oriented toward an end 50 from an opposite end 52 could force a first flange 54 to bend backward, as shown in FIG. 7. The backstop 58 acts as a buttress and keeps this from happening.

Also shown, the first flange 54 has an expanded first flange configuration 130 that defines an expanded first flange diameter 140 and a constrained first flange configuration 134 that defines a constrained first flange diameter 136. The second flange 56 has an expanded second flange configuration 154 that defines an expanded second flange diameter 156, and a constrained second flange configuration 158 that defines a constrained second flange diameter 160. The expanded first flange diameter 140 may be lesser or great than the expanded second flange diameter 156. Likewise, the constrained first flange diameter 136 may be lesser or greater than the constrained second flange diameter 160. The cross-section of first or second flange constrained or unconstrained configurations do not necessarily form a circle.

The first flange 52 and/or second flange 54 may be formed as separate pieces and subsequently attached to the tube 12, or molded in place on the tube 12.

To the extent that backstops 52 are placed on the end 50 and opposite end 52, with reference to FIGS. 5A and 5B, the expanded first backstop diameter 140 may be lesser or greater than an expanded second backstop diameter 172. Likewise, the constrained first backstop diameter 144 may be lesser or greater than the constrained second backstop diameter 176. A second backstop 52 attached to the opposite end 52 may have an expanded second backstop configuration 170 and a constrained second backstop configuration 174. Of course backstops 52 may be provided on only one end of the tube 12.

The first flange 54 and second flange 56 have been shown perpendicular to the longitudinal axis 16. However, other angles are possible. Referring now to FIGS. 8A and 8B, a first flange 54 may be attached to an end 50 of the tube and a backstop 58 may be attached to the end 50 of the tube 12 and positioned to resist the first flange 54 from bending backward away from an opposite end of the tube such as opposite end 52, the first flange 54 and the backstop 58 oriented radially outward and away from an opposite end of the tube, as previously described. An advantage to this configuration becomes evident when the stent is compressed into its delivery configuration. The backstop 58 tends to straighten in response to a constraining force, which greatly facilities compressing the stent into a delivery configuration. Similarly the barb 48 tends to straighten when compressed into its delivery configuration, albeit in a direction opposite to the backstop 58.

Stents may be compressed with an iris type stent compressor. An uncompressed stent is placed in the iris and the iris is subsequently closed. This applies an inward compressive force to the stent, toward the longitudinal axis and generally perpendicular thereto. One example of a stent compressor is disclosed by US8474122B2 “Apparatus for compressing an expandable medical device” to Melsheimer.

Referring to FIG. 9, longitudinal movement of the reinforcement wire 30 is constrained. In the constrained tube configuration the wire forms loops 70 to take up the slack and the loops 70 may extend within the wall 74 and/or outside the wall 74 as indicated by arrow 71. The benefit to form a loop 70 is even greater if the reinforcement wire 30 is woven into the braid 72 because the ability of the reinforcement wire 30 to move longitudinally is even more restricted.

Referring now to FIGS. 10 and 11, elements from FIGS. 1, 2, 4A, 4B, 5A, 5B, 6A and 6B have been combined. The stent 10 comprises the tube 12. The constrainable covering 40 covers the braid 72. The end 50 and the opposite end 52 have the first flange 54 and the second flange 56, respectively. The end 50 and the opposite end 52 both have backstops 58. Only the opposite end 52 has barbs 48. Referring to FIG. 11, the tube 12 has a constrained tube configuration with the decreased first angle 23 and the decreased second angle 25 and that defines a constrained tube diameter 34 for delivery to the anatomical passageway, the constrained tube diameter 34 being less than the expanded tube diameter 14.

Backstops 58 may be bent back away from the tube 12 generally aligned with the horizontal axis 16, and the barbs 48 may be bent forward toward the tube 12 generally aligned with the horizontal axis. This could be reversed, with the backstops 58 bent forward toward the tube 12 and generally aligned with the horizontal axis 16, and the barbs 48 bent back away from the tube 12 generally aligned with the horizontal axis 16. The elements of this paragraph may be implemented with the elements of FIGS. 1, 2, 3A, 4A, 4B, 5A, 5B, 6A, 6B, 8A, 8B, 10 and 11, alone or in addition to one another.

Any stent 10 can have structure in addition to the tube 12 including elements of FIGS. 3A through 8B alone or in addition to one another. All such structure may be constrained to the extent necessary, by a stent sheath for example, to be able to deliver the stent 10 percutaneously to a location within an anatomical passageway.

The stents disclosed herein may be used in various parts of the anatomy, for example the peripheral veins and arteries, or the esophagus, but not limited to these examples. The stent 10 shown in FIG. 10 may be useful for a Transjugular Intrahepatic Portosystemic Shunt (TIPS). It is covered and may be used to shunt blood from the hepatic vein 104 to the portal vein 102 so it bypasses the liver 100. This example may be implemented with the method described with reference to FIGS. 13 through 20, although the method is not limited to this example since other examples described herein (Example 1 and Example 2) may also be implemented, or combinations of elements thereof, as described herein and evident therefrom.

Of course other combinations are possible and evident from the description provided herein.

Referring now to FIGS. 13-20, and specifically FIG. 13, a liver 100 and a portal vein 102 and hepatic vein 104 are presented. A desired path 102 for a passageway 106 is shown. The passageway 106 may be formed by penetrating the liver 100 from the hepatic vein 104 to the portal vein 102 with a needle 110, as shown in FIG. 14. The needle 110 is housed within a needle catheter 112 and extended when it reaches a desired location in the hepatic vein 104. Referring now to FIG. 15, a balloon catheter 114 may be inserted into the passageway 106 and expanded to the balloon's full diameter 116 in order to widen the passageway 106. The balloon catheter 114 is then removed from the passageway 106. The needle 110 including the needle catheter 112 and balloon catheter 114 enter the anatomy percutaneously through the jugular vein and are guided to the hepatic vein 104 as is known in the minimally invasive medical treatment art.

As shown in FIG. 16, placing the stent 10 within the passageway 106 may be done with a stent catheter 118 the stent 10 being constrained within the stent sheath 120. The stent 10 and stent catheter 118 including the stent sheath 120 also enter the anatomy percutaneously through the jugular vein and are guided to the hepatic vein 104.

Another step in the procedure after the stent 10 is placed in the passageway is to insert a balloon catheter into the passageway 106 inside the stent 10. The balloon catheter is then inflated, which ensures that the stent 10 is fully expanded and firmly seated within the passageway 106 against the liver tissue.

The stent 10 may be constrained within the stent catheter 118 by a stent sheath 120. As shown in FIG. 17, the stent 10 may be deployed in the passageway 106 by positioning the stent catheter 118 in the passageway 10 and pulling the stent sheath 120 back toward the hepatic vein 104. The stent 10 self-expands as the stent sheath 120 is withdrawn thereby removing the constraining force. As previously described with reference to FIG. 18, the stent 10 is pulled toward the hepatic vein 104 to implant the barbs 48 in the portal vein 102 and stretch the tube 12. FIGS. 19 and 20 show the liver 100 after the stent 10 is deployed and the stent catheter 118 is removed, respectively.

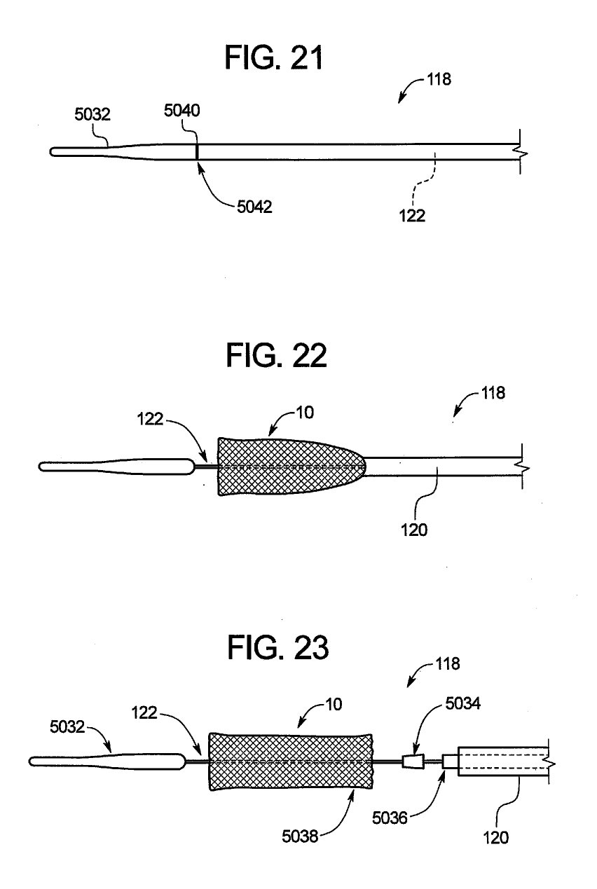

An example embodiment of a stent catheter 118 is shown in FIG. 21 for use with the stent 10 that is loaded onto the delivery stent catheter 118. In FIG. 21 the stent 10 is fully loaded into the stent catheter 118 with a distal end 5040 of the stent sheath 120 abutting the distal tip 5032 of the inner catheter 122 to form a smooth outer surface 5042.

In FIG. 22, the stent 10 is shown in a partially expanded tube configuration 60 around the inner catheter 122 and positioned between a distal tip 5032 and the distal end 5040 by partially removing the stent sheath 120 from the stent 10. The proximal end of the stent is held within the stent sheath 120. A handle moves the stent sheath 120 relative to the inner catheter 122. The stent sheath 120 is pulled proximally by the handle to expose the stent 10 at the patient delivery site.

A fully expanded stent is presented in FIG. 23 along with additional details of the stent catheter 118. The stent catheter may include a pusher catheter 5036 proximal to the holder device 5034. A proximal portion 5038 of the stent 10 may be collapsed around the holder device 5034 and held in place by the stent sheath 120 (see FIG. 22). The stent 10 is captured between the holder device 5034 and the stent sheath 120.

In operation of the stent catheter 118, the holder device 5034 may be used to hold the proximal portion 5038 of the stent 5004 between the inner catheter 122 and the stent sheath 120 (see FIG. 22). The stent 10 may be re-sheathed with the stent sheath 120 when up to about 90-95% of the stent 10 has been unsheathed. The holder device 5034 may be used to retain the remaining 5-10% of the proximal portion 5038 of the stent 10 under constraint by the stent sheath 120. Furthermore, this arrangement may hold the stent 10 within the stent sheath 120 when it is pulled during a TIPS procedure as previously described with reference to FIGS. 17 and 18 where the stent 10 is placed in the passageway 106 by partially deploying the end 50 of the tube 12 with the a barb 48 into the portal vein 102, and then pulling the tube 12 toward the hepatic vein 104 to implant the barb 48 in the portal vein 102 and to stretch the tube 12.

All features and modifications of the described examples and dependent claims are usable in all aspects of the examples taught herein. Furthermore, the individual features of the dependent claims, as well as all features and modifications of the described examples are combinable and interchangeable with one another.

What is claimed is:

1. A stent for placement in an anatomical passageway, comprising:
a self-expanding tube having an expanded tube configuration that defines an expanded tube diameter and a longitudinal axis,
the tube comprising a first wire and at least a second wire both helically positioned along the longitudinal axis forming a braid at the expanded tube diameter,
the first wire comprising first wire portions, the second wire comprising second wire portions,
the first wire portions being oriented at an acute first angle clockwise relative to the longitudinal axis,
the second wire portions being oriented at an acute second angle counter-clockwise relative to the longitudinal axis,
a reinforcement wire helically positioned along the longitudinal axis and adjacent the first wire portions and the second wire portions,
the reinforcement wire comprising a third wire portion oriented at an acute third angle relative to the longitudinal axis, and a fourth wire portion adjacent to the third wire portion and oriented at an acute fourth angle relative to the longitudinal axis that is in the same direction as the third angle and is less than the third angle, the third angle being greater than the first angle and the second angle; and,
the tube having a constrained configuration with a decreased first angle and a decreased second angle and that defines a constrained tube diameter for delivery to the anatomical passageway, the constrained tube diameter being less than the expanded tube diameter.

2. The stent of claim 1, the reinforcement wire being woven into the braid. 5

3. The stent of claim 1, comprising a first z-stent attached to an end of the tube and a second z-stent attached to an opposite end of the tube, the first z-stent and the second z-stent being self-expanding, the first z-stent having an expanded first z-stent configuration that defines an expanded first z-stent diameter and a constrained first z-stent configuration that defines a constrained first z-stent diameter, the second z-stent having an expanded second z-stent configuration that defines an expanded second z-stent diameter and a constrained second z-stent configuration that defines a constrained second z-stent diameter, the constrained first z-stent diameter being less than the expanded first z-stent diameter and the constrained second z-stent diameter being less than the expanded second z-stent diameter.

4. The stent of claim 1, comprising a first barb attached to an end of the tube and oriented radially outward toward an opposite end of the tube, the first barb having an expanded first barb configuration that defines an expanded first barb diameter and a constrained first barb configuration that defines a constrained first barb diameter, the constrained first barb diameter being less than the expanded first barb diameter.

5. The stent of claim 1, comprising a first flange attached to an end of the tube and a first backstop attached to the end of the tube and positioned to resist the first flange from bending backward away from an opposite end of the tube, the first flange having an expanded first flange configuration that defines an expanded first flange diameter and a constrained first flange configuration that defines a constrained first flange diameter, the constrained first flange diameter being less than the expanded first flange diameter, the first backstop having an expanded first backstop configuration that defines an expanded first backstop diameter, and a constrained first backstop configuration that defines a constrained first backstop diameter, the constrained first backstop diameter being less than the expanded first backstop diameter.

6. The stent of claim 1, comprising a first flange attached to an end of the tube and a first backstop attached to the end of the tube and positioned to resist the first flange from bending backward away from an opposite end of the tube, the first flange and the first backstop oriented radially outward and away from an opposite end of the tube, the first flange having an expanded first flange configuration that defines an expanded first flange diameter and a constrained first flange configuration that defines a constrained first flange diameter, the constrained first flange diameter being less than the expanded first flange diameter, the first backstop having an expanded first backstop configuration that defines an expanded first backstop diameter, and a constrained first backstop configuration that defines a constrained first backstop diameter, the constrained first backstop diameter being less than the expanded first backstop diameter.

7. A stent for placement in an anatomical passageway, comprising:
a self-expanding tube having an expanded tube configuration that defines a expanded tube diameter and a longitudinal axis,
the tube comprising a first wire and at least a second wire both helically positioned along the longitudinal axis forming a braid at the expanded tube diameter,
the first wire comprising first wire portions, the second wire comprising second wire portions,
the first wire portions being oriented at an acute first angle clockwise relative to the longitudinal axis,
the second wire portions being oriented at an acute second angle counter-clockwise relative to the longitudinal axis,
a reinforcement wire helically positioned along the longitudinal axis and adjacent the first wire portions and the second wire portions,
at least a portion of the reinforcement wire being oriented at an acute third angle relative to the longitudinal axis,
the third angle being greater than the first angle and the second angle,
the tube having a constrained tube configuration with a decreased first angle and a decreased second angle and that defines a constrained tube diameter for delivery to the anatomical passageway, the constrained tube diameter being less than the expanded tube diameter, and the reinforcement wire forming a loop in the constrained tube configuration that does not exist in the expanded tube configuration.

8. The stent of clam 7, the braid defining a wall, the loop extending within the wall.

9. The stent of claim 7, the braid defining a wall, the loop extending outside the wall.

10. The stent of claim 7, the reinforcement wire comprising a third wire portion oriented at the acute third angle relative to the longitudinal axis, and a fourth wire portion adjacent to the third wire portion and oriented at an acute fourth angle relative to the longitudinal axis that is in the same direction as the third angle and is less than the third angle, the third angle being greater than the first angle and the second angle.

11. The stent of claim 7, the reinforcement wire being woven into the braid.

12. The stent of claim 7, comprising a first z-stent attached to an end of the tube and a second z-stent attached to an opposite end of the tube, the first z-stent and the second z-stent being self-expanding, the first z-stent having an expanded first z-stent configuration that defines an expanded first z-stent diameter and a constrained first z-stent configuration that defines a constrained first z-stent diameter, the constrained first z-stent diameter being less than the expanded first z-stent diameter, the second z-stent having an expanded second z-stent configuration that defines an expanded second z-stent diameter and a constrained second z-stent configuration that defines a constrained second z-stent diameter, the constrained second z-stent diameter being less than the expanded second z-stent diameter.

13. The stent of claim 7, comprising a first barb attached to an end of the tube and oriented radially outward toward an opposite end of the tube, the first barb having an expanded first barb configuration that defines an expanded first barb diameter and a constrained first barb configuration that defines a constrained first barb diameter, the constrained first barb diameter being less than the expanded first barb diameter.

14. The stent of claim 7, comprising a first flange attached to an end of the tube and a first backstop attached to the end of the tube and positioned to resist the first flange from bending backward away from an opposite end of the tube, the first flange having an expanded first flange configuration that defines an expanded first flange diameter and a constrained first flange configuration that defines a constrained first flange diameter, the constrained first flange diameter being less than the expanded first flange diameter, the first backstop having an expanded first backstop configuration that defines an expanded first backstop diameter, and a constrained first backstop configuration that defines a constrained first backstop diameter, the constrained first backstop diameter being less than the expanded first backstop diameter.

15. The stent of claim 7, comprising a first flange attached to an end of the tube and a first backstop attached to the end of the tube and positioned to resist the first flange from bending backward away from an opposite end of the tube, the first flange and the first backstop oriented radially outward and away from an opposite end of the tube, the first flange having an expanded first flange configuration that defines an expanded first flange diameter and a constrained first flange configuration that defines a constrained first flange diameter, the constrained first flange diameter being less than the expanded first flange diameter, the first backstop having an expanded first backstop configuration that defines an expanded first backstop diameter, and a constrained first backstop configuration that defines a constrained first backstop diameter, the constrained first backstop diameter being less than the expanded first backstop diameter.

16. A stent for placement in an anatomical passageway, comprising:
a self-expanding tube having an expanded tube configuration that defines an expanded tube diameter and a longitudinal axis,
the tube comprising a first wire and at least a second wire both helically positioned along the longitudinal axis forming a braid at the expanded tube diameter,
the first wire comprising first wire portions, the second wire comprising second wire portions,
the first wire portions being oriented at an acute first angle clockwise relative to the longitudinal axis,
the second wire portions being oriented at an acute second angle counter-clockwise relative to the longitudinal axis,
a reinforcement wire helically positioned along the longitudinal axis and adjacent the first wire portions and the second wire portion,
the reinforcement wire comprising a third wire portion oriented at an acute third angle relative to the longitudinal axis, and a fourth wire portion adjacent to the third wire portion and oriented at an acute fourth angle relative to the longitudinal axis that is in the same direction as the third angle and is less than the third angle, the third angle being greater than the first angle and the second angle; and,
the tube having a constrained configuration with a decreased first angle and a decreased second angle and that defines a constrained tube diameter for delivery to the anatomical passageway, the constrained tube diameter being less than the expanded tube diameter; and,
a first z-stent overlapping an end of the tube, the first z-stent being self-expanding and contiguous with the tube, the first z-stent having an expanded first z-stent configuration that defines an expanded first z-stent diameter and a constrained first z-stent configuration that defines a constrained first z-stent diameter, the constrained first z-stent diameter being less than the expanded first z-stent diameter;
a second z-stent overlapping an opposite end of the tube, the second z-stent being self-expanding and contiguous with the tube, the second z-stent having an expanded second z-stent configuration that defines an expanded second z-stent diameter and a constrained second z-stent configuration that defines a constrained second z-stent diameter, the constrained second z-stent diameter being less than the expanded second z-stent diameter;
a first flange contiguous with the first z-stent, the first flange having an expanded flange configuration that defines an expanded first flange diameter and a constrained first flange configuration that defines a constrained first flange diameter, the constrained first flange diameter being less than the expanded first flange diameter;
a second flange contiguous with the second z-stent, the second flange having an expanded second flange configuration that defines an expanded second flange diameter and a constrained second flange diameter that defines a constrained second flange diameter, the constrained second flange diameter being less than the expanded second flange diameter;
a first backstop contiguous with the first z-stent positioned to resist the first flange from bending backward away from the opposite end of the tube, the first backstop having an expanded first backstop configuration that defines an expanded first backstop diameter, and a constrained first backstop configuration that defines a constrained first backstop diameter, the constrained first backstop diameter being less than, the expanded first backstop diameter;
a second backstop contiguous with the second z-stent positioned to resist the second flange from bending backward away from the end of the tube, the second backstop having an expanded configuration that defines an expanded second backstop diameter, and the constrained second backstop configuration that defines a constrained second backstop diameter, the constrained second backstop diameter being less than the expanded second backstop diameter;
a first barb contiguous with the first z-stent and oriented radially outward toward the opposite end of the tube, the first barb having an expanded first barb configuration that defines an expanded first barb diameter and a constrained first barb configuration that defines a constrained first barb diameter, the constrained first barb diameter being less than the expanded first barb diameter; and,
a constrainable covering layer contiguous with the tube that covers at least the tube that is not covered by the first flange and the second flange.

17. A method of stenting in a liver between a portal vein and a hepatic vein, comprising:
percutaneously accessing the hepatic vein;
forming a passageway in the liver between the portal vein and the hepatic vein; placing a self-expanding tube in the passageway,
the self-expanding tube having an expanded configuration that defines an expanded tube diameter and a longitudinal axis,
the tube comprising a first wire and at least a second wire both helically positioned along the longitudinal axis forming a braid at the expanded tube diameter,
the first wire comprising first wire portions, the second wire comprising second wire portions,
the first wire portions being oriented at an acute first angle clockwise relative to the longitudinal axis,
the second wire portions being oriented at an acute second angle counter-clockwise relative to the longitudinal axis,
a reinforcement wire helically positioned along the longitudinal axis and adjacent the first wire portions and the second wire portions,
the reinforcement wire comprising a third wire portion oriented at an acute third angle relative to the longitudinal axis, and a
fourth wire portion adjacent to the third wire portion and oriented at an acute fourth angle relative to the longitudinal axis that is in the same direction as the third angle and is less than the third angle, the third angle being greater than the first angle and the second angle; and,
the tube having a constrained configuration with a decreased first angle and a decreased second angle and that defines a constrained tube diameter for delivery to the anatomical passageway, the constrained tube diameter being less than the expanded tube diameter, the tube being in the constrained tube configuration within a stent sheath; and,
expanding from the constrained tube configuration to the expanded tube configuration by removing the stent sheath from the tube, and leaving the tube within the passageway.

18. The method of claim 17, the tube comprising a first barb attached to an end of the tube and oriented radially outward toward an opposite end of the tube, the first barb having an expanded first barb configuration that defines an expanded first barb diameter and a constrained first barb configuration that defines a constrained first barb diameter, the constrained first barb diameter being less than the expanded first barb diameter, the first barb being in the constrained first barb configuration within the stent sheath, the method comprising:
partially expanding the end of the tube and fully expanding the first barb and the first flange into the portal vein by partially removing the stent sheath from the tube;
pulling the tube toward the hepatic vein to implant the barb in the portal vein and to stretch the tube; and,
expanding the rest of the tube with the second flange in the hepatic vein by completely removing the stent sheath from the tube.

19. The method of claim 17, the tube comprising
a first barb attached to an end of the tube and oriented radially outward toward an opposite end of the tube, the first barb having an expanded first barb configuration that defines an expanded first barb diameter and a constrained first barb configuration that defines a constrained first barb diameter, the constrained first barb diameter being less than the expanded first barb diameter, the first barb being in the constrained first barb configuration within the stent sheath, and
a first flange attached to an end of the tube and a second flange attached to an opposite end of the tube, the first flange having an expanded first flange configuration that defines an expanded first flange diameter, and the first flange having a constrained first flange configuration that defines a constrained first flange diameter, the constrained first flange diameter being less than the expanded first flange diameter, the first flange being in the constrained first flange configuration within the stent sheath, and
the second flange having an expanded second flange configuration that defines an expanded second flange diameter and a constrained second flange configuration that defines a constrained second flange diameter, the constrained second flange diameter being less than the expanded second flange diameter, the second flange being in the constrained second flange configuration within the stent sheath, and
a constrainable covering layer attached to the tube that covers at least the tube that is not covered by the first flange and the second flange, the constrainable covering being constrained within the stent sheath, the method comprising:
partially expanding the end of the tube and fully expanding the first barb and the first flange into the portal vein so the tube is partially in the expanded tube configuration and the first barb is in the expanded first barb configuration and the first flange is in the expanded first flange configuration by partially removing the stent sheath from the tube;
pulling the tube toward the hepatic vein to implant the first barb in the portal vein and to stretch the tube; and,
expanding the rest of the tube with the second flange in the hepatic vein, the tube being in its expanded tube configuration within the passageway and the second flange being in its expanded second flange configuration by completely removing the stent sheath from the tube.

20. The method of claim 17, comprising a first flange attached to an end of the tube and a first backstop attached to the end of the tube and positioned to resist the first flange from bending backward away from an opposite end of the tube, the first flange and the first backstop oriented radially outward and away from an opposite end of the tube, the first flange having an expanded first flange configuration that defines an expanded first flange diameter and a constrained first flange configuration that defines a constrained first flange diameter, the constrained first flange diameter being less than the expanded first flange diameter, the first flange being in the constrained first flange configuration, the first backstop having an expanded first backstop configuration that defines an expanded first backstop diameter, and a constrained first backstop configuration that defines a constrained first backstop diameter, the constrained first backstop diameter being less than the expanded first backstop diameter, the first flange being in the constrained first flange configuration and the first backstop being in the constrained first backstop configuration prior to the step of expanding the rest of the tube in the passageway so the tube is in the expanded tube configuration by removing the tube from the stent sheath.
